(19) United States
(12) Patent Application Publication
(10) Pub. No.: US 2011/0247631 A1
(43) Pub. Date: Oct. 13, 2011

(54) FULL MALE PROTECTION PROPHYLACTIC SYSTEMS

(76) Inventor: Joseph Cornell, Columbia, SC (US)

(21) Appl. No.: 13/081,579

(22) Filed: Apr. 7, 2011

Related U.S. Application Data
(60) Provisional application No. 61/321,930, filed on Apr. 8, 2010.

(51) Int. Cl. A61F 6/02

(52) U.S. Cl. 128/842

(57) ABSTRACT

A full male protection prophylactic system comprising an anatomically-male configured rubberized skin protector having a penis-insertible slit, a pair of male prophylactic support stabilizer, and an adjustable waist band wearable by a male-user and providing maximum protection to the pubic skin area surrounding the penis from skin-to-skin sexually transmitted diseases during sexual intercourse. The anatomically-male configured rubberized skin protector comprises a latex material and provides protection to the male-user's inner thighs, scrotum, testicles, and penis shaft. First and second male prophylactic support stabilizers each comprise a plurality of flexible, sturdy rods that may serve to sustain relative placement of the anatomically-male configured rubberized skin protector to the pubic skin surface during intercourse. The waist band may be elastically deformable thereby providing size adjusting means for the male-user.

### CROSS-REFERENCE TO RELATED APPLICATION

The present application is related to and claims priority from prior provisional application Ser. No. 61/321,930, filed Apr. 8, 2010 which application is incorporated herein by reference.

### BACKGROUND OF THE INVENTION

1. Field of the Invention

The present invention relates generally to the field of male prophylactics and more specifically relates to a latex prophylactic worn by a male during sexual intercourse that may protect the inner thighs and the skin surrounding the male genitals against sexually transmitted diseases that are spread through skin to skin contact.

2. Description of the Related Art

Sexually transmitted diseases are a serious concern to any man or woman that is sexually active. If a person has an STD and is too embarrassed to tell their partner, the partner is then at risk for contracting the disease. Sometimes, people are unaware that they are carrying a disease and can pass it on to other people unknowingly. Condoms are useful for preventing against pregnancy and HIV but they do very little to protect against diseases that are spread through skin-to-skin contact. Chlamydia, genital herpes, genital lice, genital warts, gonorrhea, hepatitis, scabies, and syphilis are all examples of venereal diseases that may be spread through skin to skin contact.

Most conventional prophylactics are effective for preventing the exchange of fluid during intercourse; however conventional prophylactics, such as condoms, fail to protect the skin surrounding the male member from exposure to disease. More specifically, the inner thigh, scrotum, and pubic regions are areas of skin which may be exposed to disease during sexual interaction. A more effective prophylactic is needed.

Various attempts have been made to solve the above-mentioned problems such as those found in U.S. Pat. Nos. 4,993,431, 4,993,433, 5,535,757, and 2,591,783. This prior art is representative of male prophylactics for use during intercourse. None of the above inventions and patents, taken either singly or in combination, is seen to describe the invention as claimed.

Ideally, a male prophylactic should be user-friendly, yet would operate reliably and be manufactured at a modest expense. Thus, a need exists for a reliable male prophylactic system to be worn by a male during sexual intercourse that may provide additional protection against sexually transmitted diseases spreadable via skin to skin contact and to avoid the above-mentioned problems.

### BRIEF SUMMARY OF THE INVENTION

In view of the foregoing disadvantages inherent in the known male prophylactics art, the present invention provides a novel full protection male prophylactics system. The general purpose of the present invention, which will be described subsequently in greater detail, is to provide a reliable latex male prophylactic to be worn by a male during sexual intercourse that may cover the skin surrounding the male penis vulnerable to contracting sexually transmitted diseases which are spread through skin to skin contact.

A full protection male prophylactic system is disclosed herein and may comprise an anatomically-male configured rubberized skin protector, at least one first male prophylactic support stabilizer, at least one second male prophylactic support stabilizer, and a waist band. The anatomically-male configured rubberized skin protector serves to protect a pubic skin surface from contact during intercourse. The first and second male prophylactic support stabilizers may serve to sustain relative placement of the anatomically-male configured rubberized skin protector to the pubic skin surface. The waist band may secure the full protection male prophylactic system to the male-user during sexual intercourse.

Further, the anatomically-male configured rubberized skin protector of the present invention may comprise a penis-insertable slit such that the male-user may insert his penis therethrough when the full male protection prophylactic system is worn. The anatomically-male configured rubberized skin protector may further comprise a testicle sack that is preferably non-restricting thereby providing a looser fit in the scrotum area.

The present invention may be securably attached to a male-user via a plurality of fasteners that may be attached to the anatomically-male configured rubberized skin protector. In use, the first and second male prophylactic support stabilizers may each comprise several male snappable buttons that may be snappably fastened to a plurality of female snappable buttons affixed to an exterior surface of the anatomically-male configured rubberized skin protector. The first and second male prophylactic support stabilizers may comprise supporters comprising flexible rods. The flexible rods of the present invention are preferably body-contoured such that the full protection male prophylactic system is securably wearable during sexual intercourse to the male-user.

The present invention may further comprise an adjustable waist band comprising a first waist strap and a second waist strap. The first and second waist strap may each be attached to and end of the first and second male prophylactic support stabilizers. The straps may comprise a fastening means such as a button or a buckle. The device may comprise a kit. A method of use is also described subsequently.

The present invention holds significant improvements and serves as a full protection male prophylactics system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, full male protection prophylactic system, constructed and operative according to the teachings of the present invention.

FIG. 1 shows a perspective view illustrating a full male protection prophylactic system in an in-use condition according to an embodiment of the present invention.

FIG. 2 is a perspective view illustrating an adjustable strap used to secure the full male protection prophylactic system in an in-use condition around the genitalia area of a male according to an embodiment of the present invention of FIG. 1.

FIG. 3 is a side perspective view illustrating the full male protection prophylactic system in an in-use condition according to an embodiment of the present invention of FIG. 1.

FIG. 4 is a side perspective view illustrating the full male protection prophylactic system comprising a strap fastener according to an embodiment of the present invention of FIG. 1.

FIG. 5 is a front perspective view illustrating the full male protection prophylactic system comprising first and second male prophylactic support stabilizers according to an embodiment of the present invention of FIG. 1.

FIG. 6 is a flowchart illustrating a method of use according to an embodiment of the present invention of FIGS. 1-5.

The various embodiments of the present invention will hereinafter be described in conjunction with the appended drawings, wherein like designations denote like elements.

### DETAILED DESCRIPTION

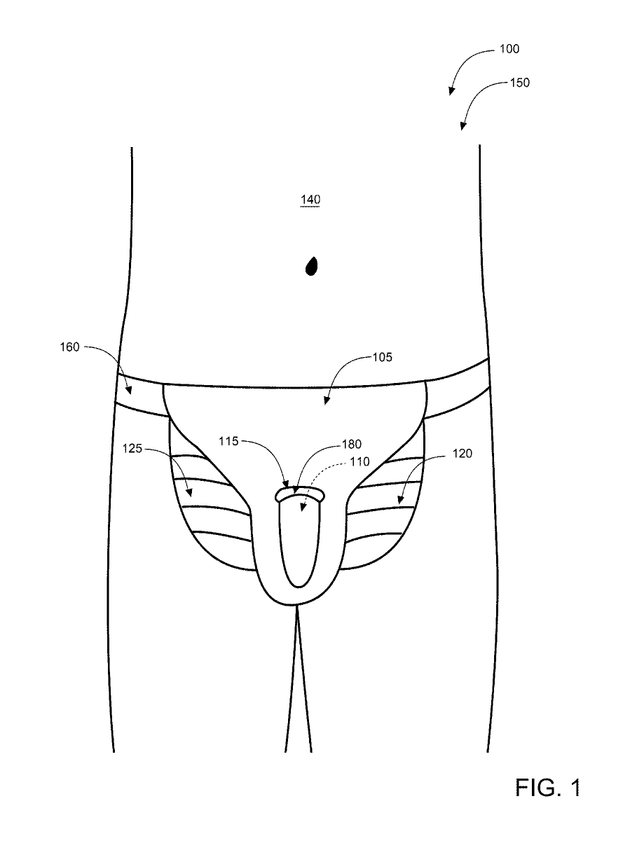

As discussed above, embodiments of the present invention relate to a full male protection prophylactic system and more particularly to a male prophylactic to be worn by a male during sexual intercourse that may provide additional protection against sexually transmitted diseases spreadable via skin to skin contact. Referring to the drawings by numerals of reference there is shown in FIG. 1, full male protection prophylactic systems 100 in an in-use condition 150 being worn by male-user 140. As shown, full male protection prophylactic systems 100 serves as a safer and more complete male prophylactic to be worn in conjunction with condom 180 providing maximum protection from sexually transmitted disease.

FIG. 1 is a front perspective view of full male protection prophylactic systems 100 comprising anatomically-male configured rubberized skin protector 105, first male prophylactic support stabilizer 120, second male prophylactic support stabilizer 125, and waist band 160 having adjustable fastening means. In a preferred embodiment, anatomically-male configured rubberized skin protector 105 comprises latex material that may be adaptably suited to conform to the skin area surrounding penis 110 such as the inner thigh, testicle sack, pubic region, and scrotum. Further, anatomically-male configured rubberized skin protector 105 may comprise penis-insertible slit 115 thereby allowing male-user 140 to insert penis 110 therethrough. As seen in FIG. 1, penis-insertible slit 115 permits just enough room for penis 180 to pass through anatomically-male configured rubberized skin protector 105. It should be appreciated that penis-insertible slit 115 may comprise double-bonded latex to prevent unintentional rippage of anatomically-male configured rubberized skin protector 105. For complete protection, once male-user 140 applies condom 180 to penis 110, full male protection prophylactic systems 100 may be positioned such that condom 180 rests underneath the flaps of penis-insertible slit 115. As shown in FIG. 1, waist band 160 may loop through anatomically-male configured rubberized skin protector 105 thereby securing full male protection prophylactic systems 100 to male-user 140.

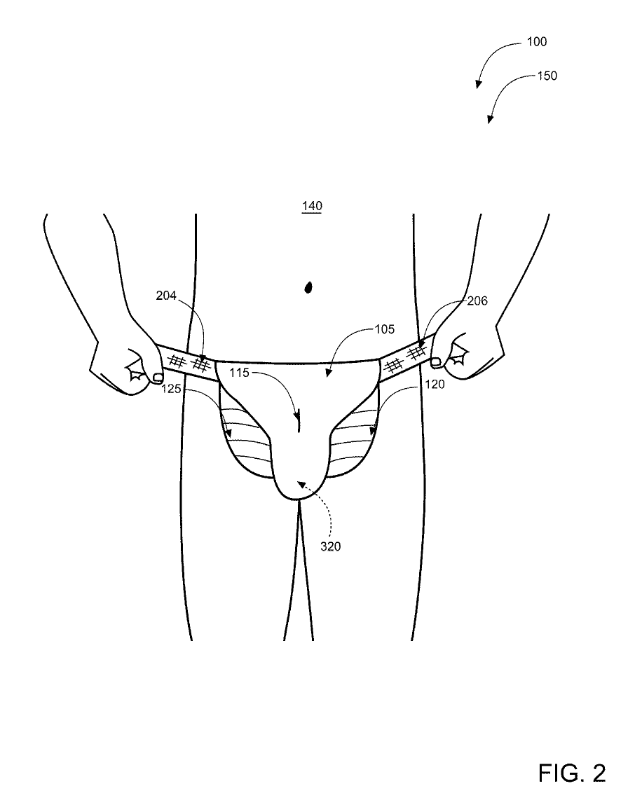

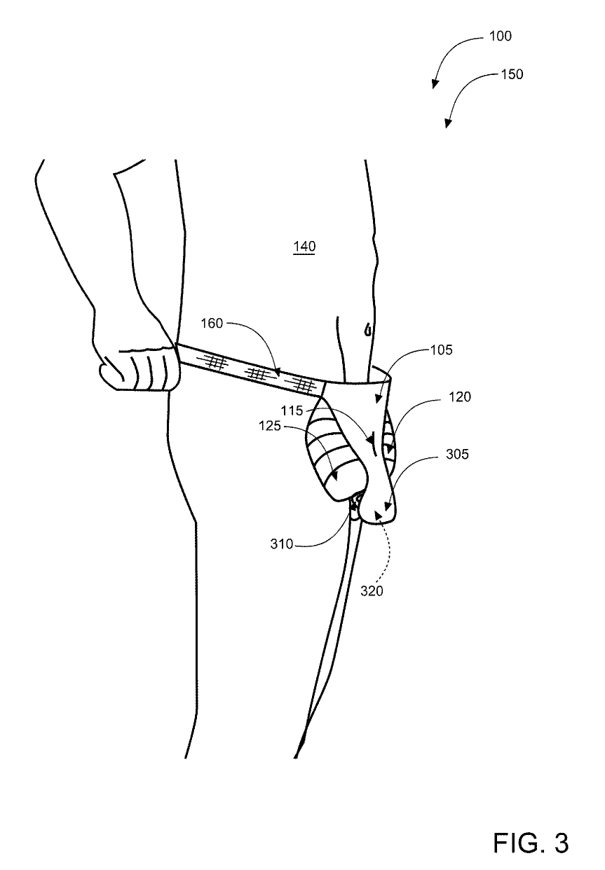

Referring now to FIGS. 2 and 3 showing front and side perspective views of full male protection prophylactic systems 100 in an in-use condition 150 being applied by male-user 140 according to an embodiment of the present invention of FIG. 1. Waist band 160 may comprise first waist strap 204 and second waist strap 206 wherein male-user 140 connects first waist strap 204 and second waist strap 206 at a user-preferred comfortable point via a fastening means. Preferably, first waist strap 204 and second waist strap 206 comprise elastic material thereby providing waist band 160 that may be easily adjustable and comfortably worn while in an in-use condition 150. As shown in FIG. 2, penis-insertible slit 115 may comprise a thin, vertical tear on a front portion of anatomically-male configured rubberized skin protector 105 thereby providing space for penis 110 to be inserted therethrough while protecting the skin surrounding penis 110 from skin-to-skin contact during sexual intercourse.

As seen in the side perspective view of FIG. 3, full male protection prophylactic systems 100 may further comprise testicle sack 305 secured to anatomically-male configured rubberized skin protector 105 via scrotum band 310. Testicle sack 305 may comprise latex material that may securely hold testicles of male-user 140 in a non-constricting manner thereby allowing sufficient breathing room for testicles of male-user 140 while full male protection prophylactic systems 100 is being worn.

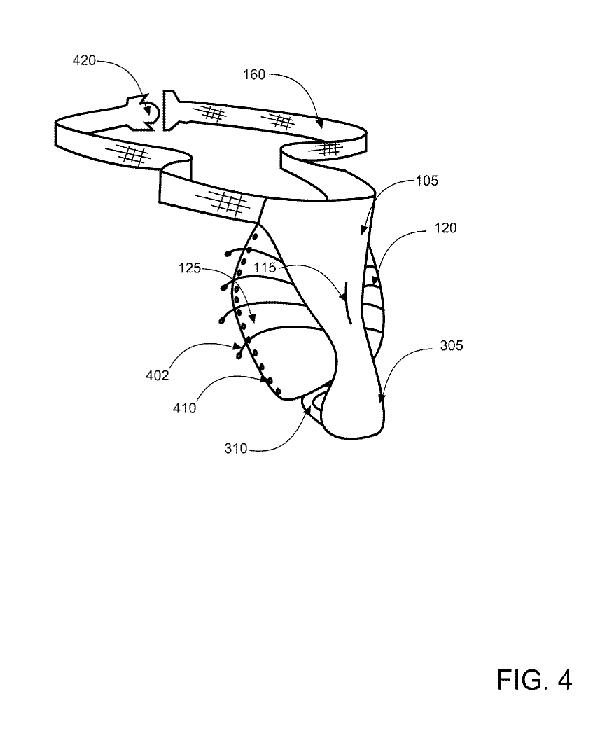

Referring now to FIG. 4 is a side perspective view illustrating full male protection prophylactic systems 100 according to an embodiment of the present invention of FIG. 1. As shown, full male protection prophylactic systems 100 may comprise first male prophylactic support stabilizer 120 and second male prophylactic support stabilizer 125 located on opposite sides of anatomically-male configured rubberized skin protector 105. First male prophylactic support stabilizer 120 and second male prophylactic support stabilizer 125 may each comprise a plurality of flexible support rods 402 connecting an inner-side portion of anatomically-male configured rubberized skin protector 105 to various contact points along an outer-side portion of anatomically-male configured rubberized skin protector 105 as well as to waist band 160.

Flexible support rods 402 may comprise a flexible, sturdy material permitting first male prophylactic support stabilizer 120 and second male prophylactic support stabilizer 125 to conform to the contours of the skin of male-user 140. To connect flexible support rods 402, a plurality of snappable buttons 410 are provided in certain embodiments. As shown in FIG. 4, a plurality of female receiving buttons may be affixed along an outer-side perimeter of anatomically-male configured anatomically-male configured rubberized skin protector 105. Attached at a distal end of each flexible support rod(s) 402 is a male connecting snap button such that the male connecting snap may be snappably connected to the female receiving button thereby securing flexible support rod(s) 402 in place. In such a manner, flexible support rod(s) 402 may be optimally positioned and securely fastened via snappable buttons 410 around thighs, scrotum, and pubic region of male-user 140. Once flexible support rod(s) are snappably fastened, first male prophylactic support stabilizer 120 and second male prophylactic support stabilizer 125 may cooperatively sustain relative placement of anatomically-male configured rubberized skin protector 105 to pubic skin surface while in an in-use condition 150. Because sexual intercourse is a very physical act often involving skin contact between partners, first male prophylactic support stabilizer 120 and second male prophylactic support stabilizer 125 serve to provide secure placement of anatomically-male configured rubberized skin protector 105 to pubic skin area surrounding penis 110 thereby eliminating skin-to-skin contact during sexual intercourse.

According to one embodiment of the present invention as seen in FIG. 4, waist band 160 may comprise clip fastener 420 serving to secure first waist strap 204 to second waist strap 206. The elastic means of waist band 160 additionally serves to provide a size-adjustable means of waist band 160.

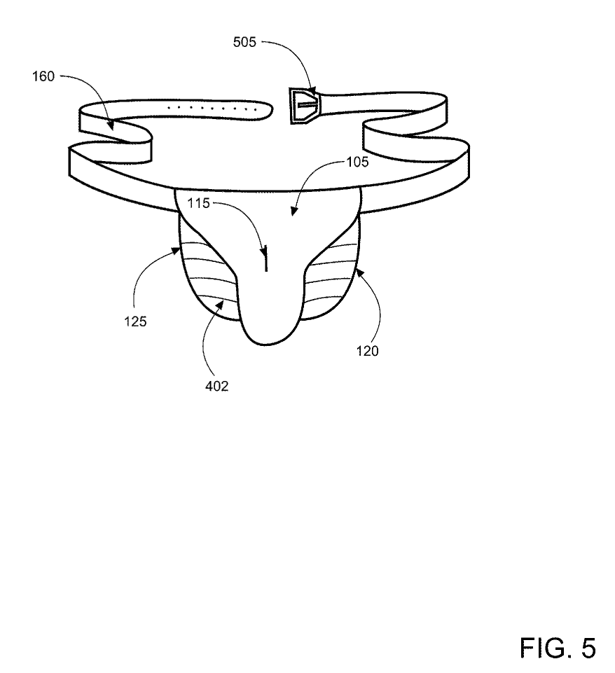

Now referring to FIG. 5, illustrating a front perspective view of full male protection prophylactic systems 100. As seen an alternative embodiment of the present invention, waist band 160 may comprise buckle fastener 505. First waist strap 204 may comprise a plurality of buckle holes and second waist strap 206 may comprise buckle fastener 505 whereby male-user 140 may select the appropriate button hole and may secure full male protection prophylactic systems 100 via buckle fastener 505.

In use, anatomically-male configured rubberized skin protector 105 is designed to protect the entire pubic region including the scrotum, penis shaft, upper leg areas, and area about penis 110 from skin contact during sexual intercourse. It should further be noted that in a preferred embodiment of the present invention, anatomically-male configured rubberized skin protector 105 is disposable after each use. It should further be noted that full male protection prophylactic systems 100 may be made available in different sizes to accommodate various body sizes and shapes of male-user 140. The adjustable nature of waist band 160 and plurality of snappable buttons 410 substantially ensures that full male protection prophylactic systems 100 may fit virtually any male-user.

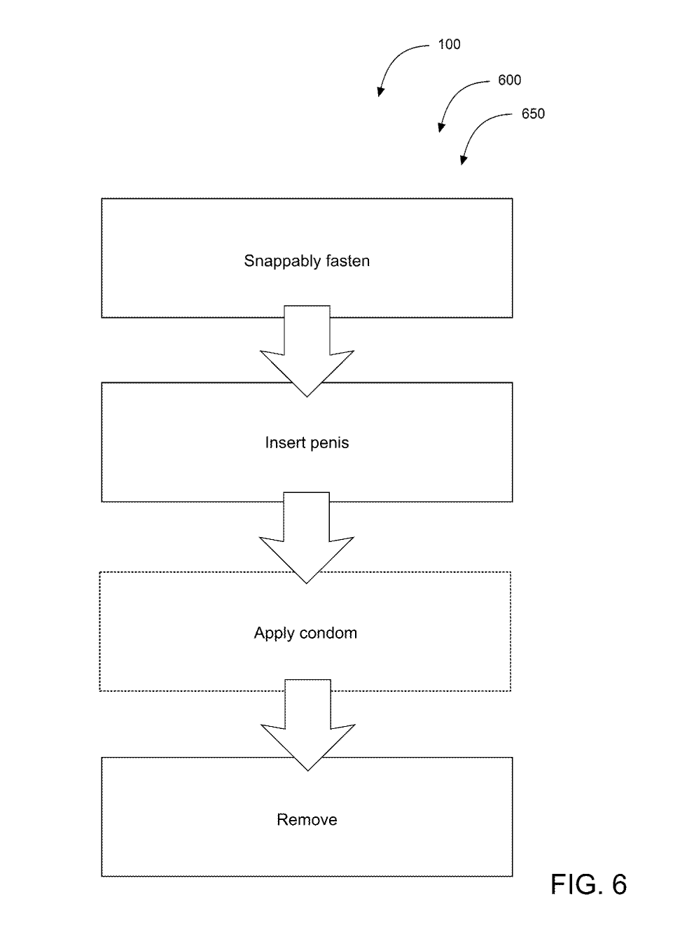

Referring now to FIG. 6, showing flowchart 650 illustrating method of use 600 for full male protection prophylactic systems 100. Method of use 600 may comprise the steps of: step one 601 snappably fastening waist band 160 around waist of male-user 140, step two 602 inserting penis 110 into penis-insertible slit 115 of anatomically-male configured rubberized skin protector 105, step three 603 applying condom 180 to penis 110, (performing intercourse) and step four 604 removing full male protection prophylactic systems 100 by unfastening waist band 160. Preferably a condom may also be applied to penis 110 in another optional step (to provide more complete isolation means—to avoid skin contact and fluid communication between user and sexual partner). It should be noted that step three 603 is an optional step and may not be implemented in all cases. Optional steps of method of use 600 are illustrated using dotted lines in FIG. 6 so as to distinguish them from the other steps of method of use 600 of full male protection prophylactic systems 100.

It should further be noted that the steps described in the method of use can be carried out in many different orders according to user preference. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A full protection male prophylactic system comprising;
an anatomically-male configured rubberized skin protector;
at least one first male prophylactic support stabilizer;
at least one second male prophylactic support stabilizer;
a waist band;
wherein said anatomically-male configured rubberized skin protector serves to protect a pubic skin surface from contact during intercourse;
wherein said first and second male prophylactic support stabilizers sustain relative placement of said anatomically-male configured rubberized skin protector to said pubic skin surface; and
wherein said waist band secures said full protection male prophylactic system to said male-user during intercourse.

2. The full protection male prophylactic system of claim 1, wherein said anatomically-male configured rubberized skin protector comprises a penis-insertable slit.

3. The full protection male prophylactic system of claim 1, wherein said anatomically-male configured rubberized skin protector further comprises a testicle sack.

4. The full protection male prophylactic system of claim 3, wherein said testicle sack is non-constricting.

5. The full protection male prophylactic system of claim 1, wherein said anatomically-male configured rubberized skin protector further comprises a plurality of fasteners.

6. The full protection male prophylactic system of claim 5, wherein said plurality of fasteners comprises at least one female receiver portion on said anatomically-male configured rubberized skin protector.

7. The full protection male prophylactic system of claim 6, wherein said plurality of fasteners comprise at least one male snappable button on said first and second male prophylactic support stabilizers.

8. The full protection male prophylactic system of claim 7, wherein said plurality of fasteners are used to removably couple said first male prophylactic support stabilizer to a left side of said anatomically-male configured rubberized skin protector, and said second male prophylactic support stabilizer to a right side of said anatomically-male configured rubberized skin protector.

9. The full protection male prophylactic system of claim 1 wherein said first and second male prophylactic support stabilizers comprise supporters.

10. The full protection male prophylactic system of claim 9 wherein said supporters comprise rods.

11. The full protection male prophylactic system of claim 10 wherein said rods are flexible.

12. The full protection male prophylactic system of claim 9 wherein said supporters are body-contoured.

13. The full protection male prophylactic system of claim 1, wherein said waist band comprises a first waist strap and a second waist strap.

14. The full protection male prophylactic system of claim 13, wherein said first waist strap is adjustably fastenable to said second waist strap to a user-preferred length.

15. The full protection male prophylactic system of claim 13, wherein said waist band is elastically deformable.

16. The full protection male prophylactic system of claim 1 further comprising a scrotum band.

17. A full protection male prophylactic system comprising;
an anatomically-male configured rubberized skin protector comprising a penis-insertable slit, a testicle sack that is non-constricting, a cushiony scrotum-band securing said testicle sack to a male scrotum, and a plurality of fasteners comprising female snappable buttons for receiving male snaps located around the outside of said anatomically-male configured rubberized skin protector;
at least one first male prophylactic support stabilizer comprising supporters comprising flexible rods;
at least one second male prophylactic support stabilizer comprising supporters comprising flexible rods;
a waist band comprising an elastically deformable first waist strap and an elastically deformable second waist strap;
wherein said anatomically-male configured rubberized skin protector comprises skin-contouring latex serving to protect a pubic skin surface from contact during intercourse;
wherein said first and second male prophylactic support stabilizers are body-contoured thereby sustaining relative placement of said anatomically-male configured rubberized skin protector to said pubic skin surface; and
wherein said waist band secures said full protection male prophylactic system to said male-user during intercourse.

18. The full protection male prophylactic system of claim 17 wherein said anatomically-male configured rubberized skin protector is available in different sizes, colors, designs, and imprinted indicia.

19. A method of using a full male protection prophylactic system comprising the steps of:
snappably fastening at least one adjustable snap around a user's waist;
inserting a penis through a penis-insertable slit;
performing intercourse; and
removing said full male protection prophylactic system.

20. The method of use of claim 19 further comprising the optional step of applying a condom to said penis after inserting said penis through said penis insertable slit.
